(19) United States
(12) Patent Application Publication
(10) Pub. No.: US 2017/0071521 A1
(43) Pub. Date: Mar. 16, 2017

(54) REPRESENTING A SUBJECT'S STATE OF MIND USING A PSYCHOPHYSIOLOGICAL MODEL

(71) Applicants: Xerox Corporation, Norwalk, CT (US); Palo Alto Research Center Incorporated, Palo Alto, CA (US)

(72) Inventors: Lalit Keshav MESTHA, Fairport, NY (US); Xuejin WEN, Fairport, NY (US); Ashish PATTEKAR, Cupertino, CA (US); Felicia LINN, San Jose, CA (US)

(21) Appl. No.: 14/853,447

(22) Filed: Sep. 14, 2015

(51) Int. Cl. A61B 5/16

(52) U.S. Cl. CPC A61B 5/165

(57) ABSTRACT

What is disclosed is a system and method for representing a subject's state of mind given a plurality of physiological inputs. In one embodiment, a vector of physiological features is received. The vector of physiological features is provided to a psychophysiological model which comprises a plurality of models which fit the physiological features to psychological quantities, each representing a different state of mind. In a manner more fully disclosed herein, the psychological quantities are then aggregated to obtain an aggregate output that is representative of the subject's overall state of mind. Once the subject's state of mind has been represented, remedial action can then be taken.

### TECHNICAL FIELD

The present invention is directed to systems and methods for representing a subject's state of mind given a plurality of physiological inputs.

### BACKGROUND

Sympathetic and Parasympathetic nervous systems are two branches of the motor Autonomic Nervous System (ANS), part of Peripheral Nervous System (PNS). These control visceral functions largely outside our awareness such as heart rate, blood pressure, etc. The sympathetic division is generally responsible for the body's excited states such as, for instance, the ‘fight or flight’ response. The parasympathetic division is generally responsible for relaxed states. States of mind such as stress, frustration, happiness, etc., are neural functions that often lead to physiological responses. Nerves of the ANS innervate various organ structures away from the brain and can elicit detectable changes in physiological parameters which may be measureable with wearable devices, such as a smart watch with appropriate sensors. These types of devices may be much easier to wear on the body than a helmet comprising of numerous EEG electrodes. Stress, frustration, joy, fatigue and anxiety are a few aspects of a person's state of mind. The human body may experience these situations due to a wide range of external stimuli. Abnormal increase, such as repeated or prolonged episodes under these situations may compromise long-term health and disrupt the body's ability to respond to events that require a quick physical response, such as quickly pulling a hand away from a hot flame or dealing effectively with an adverse situation involving interactions with another individual. For example, in a call center environment the agents often experience stress when communicating with customers (e.g., while dealing with irate customers, or when the agent's role is either in conflict or ambiguous).

Accordingly, what is needed in this art are methods for representing a subject's state of mind given a plurality of physiological inputs.

### INCORPORATED REFERENCES

The following U.S. patents, U.S. patent applications, and Publications are incorporated herein in their entirety by reference.

“Determining Arterial Pulse Transit Time From Time-Series Signals Obtained At Proximal And Distal Arterial Sites”, U.S. patent application Ser. No. 14/515,618, by Mestha et al.

“Discriminating Between Atrial Fibrillation And Sinus Rhythm In Physiological Signals Obtained From Video”, U.S. patent application Ser. No. 14/242,322, by Mestha et al.

“Determining Arterial Pulse Wave Transit Time From VPG And ECG/EKG Signals”, U.S. patent application Ser. No. 14/268,656, by Mestha et al.

“Determining Pulse Wave Transit Time From PPG And ECG/EKG Signals”, U.S. patent application Ser. No. 14/596,344, by Mestha et al.

“Method And Apparatus For Monitoring A Subject For Atrial Fibrillation”, U.S. patent application Ser. No. 13/937,740, by Mestha et al.

“Method And Apparatus For Monitoring A Subject For Functional Blood Oxygen Saturation”, U.S. patent application Ser. No. 13/937,782, by Mandel et al.

“Method And Apparatus For Monitoring A Subject For Fractional Blood Oxygen Saturation”, and U.S. patent application Ser. No. 13/937,949, by Mestha et al.

“Breathing Pattern Identification For Respiratory Function Assessment”, U.S. patent application Ser. No. 14/044,043, by Mestha et al.

“Continuous Cardiac Signal Generation From A Video Of A Subject Being Monitored For Cardiac Function”, U.S. patent application Ser. No. 13/871,766, by Kyal et al.

“Processing Source Video For Real-Time Enhancement Of A Signal Of Interest”, U.S. patent application Ser. No. 13/745,283, by Tanaka et al.

“System And Method For Producing Computer Control Signals From Breath Attributes”, U.S. patent application Ser. No. 14/257,393, Furst et al.

“Processing A Video For Respiration Rate Estimation”, U.S. patent application Ser. No. 13/529,648, Mestha et al.

“Handheld Cellular Apparatus For Volume Estimation”, U.S. patent application Ser. No. 13/920,241, Wu et al.

“Determining Cardiac Arrhythmia From A Video Of A Subject Being Monitored For Cardiac Function”, U.S. patent application Ser. No. 14/245,405, by Mestha et al.

“Generating A Flow-Volume Loop For Respiratory Function Assessment”, U.S. patent application Ser. No. 14/023,654, Mestha et al.

“Processing a Video for Tidal Chest Volume Estimation”, U.S. patent application Ser. No. 13/486,637, Bernal et al.

“Real-Time Video Processing For Respiratory Function Analysis”, U.S. patent application Ser. No. 14/195,111, Kyal et al.

“Non-Contact Monitoring Of Spatio-Temporal Respiratory Mechanics Via Depth Sensing”, U.S. patent application Ser. No. 14/223,402, Bernal et al.

“System And Method For Determining Respiration Rate From A Video”, U.S. patent application Ser. No. 14/519,641, by Mestha et al.

“Determining A Respiratory Pattern From A Video Of A Subject”, U.S. patent application Ser. No. 14/742,233, by Prasad et al.

“System And Method For Determining Video-Based Pulse Transit Time With Time-Series Signals”, U.S. patent application Ser. No. 14/026,739, by Mestha et al.

“A Video Acquisition System And Method For Monitoring A Subject For A Desired Physiological Function”, U.S. patent application Ser. No. 13/921,939, by Xu et al.

“Minute Ventilation Estimation Based On Chest Volume”, U.S. patent application Ser. No. 13/486,715, by Bernal et al.

“Minute Ventilation Estimation Based On Depth Maps”, U.S. Pat. No. 8,971,985

“Respiratory Function Estimation From A 2D Monocular Video”, U.S. Pat. No. 8,792,969

“Monitoring Respiration With A Thermal Imaging System”, U.S. Pat. No. 8,790,269

“Estimating Cardiac Pulse Recovery From Multi-Channel Source Data Via Constrained Source Separation”, U.S. Pat. No. 8,617,081

“Video-Based Estimation Of Heart Rate Variability”, U.S. Pat. No. 8,977,347

“Continuous Cardiac Pulse Rate Estimation From Multi-Channel Source Video Data”, U.S. Pat. No. 8,855,384.

“Determining Cardiac Arrhythmia From A Video Of A Subject Being Monitored For Cardiac Function”, U.S. Pat. No. 8,768,438

“Continuous Cardiac Pulse Rate Estimation From Multi-Channel Source Video Data With Mid-Point Stitching”, U.S. Pat. No. 9,036,877

“Systems And Methods For Non-Contact Heart Rate Sensing”, U.S. Pat. No. 9,020,185

### BRIEF SUMMARY

What is disclosed is a system and method for representing a subject's state of mind given a plurality of physiological inputs. In one embodiment, a vector of physiological features is received. The vector of physiological features is provided to a psychophysiological model which comprises a plurality of models which fit the physiological features to psychological quantities, each representing a different state of mind. In a manner more fully disclosed herein, the psychological quantities are then aggregated to obtain an aggregate output that is representative of the subject's overall state of mind. Once the subject's state of mind has been represented, remedial action can then be taken.

Features and advantages of the methods disclosed herein will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows schematically the effects of ANS on the heart;

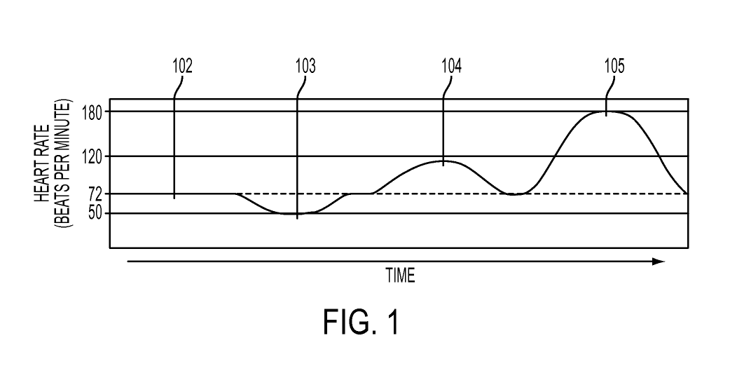

FIG. 2 shows two heart rate graphs for two different emotional states: frustration and appreciation;

FIG. 3 is a flow diagram which illustrates one example embodiment of the present method for representing a subject's state of mind from a plurality of physiological inputs; and

FIG. 4 is a functional block diagram of a special purpose for performing various aspects of the present method for representing a subject's state of mind as described with respect to the flow diagram of FIG. 3.

### DETAILED DESCRIPTION

What is disclosed is a multi-parameter data driven vectorized model comprising a multiple physiological inputs (e.g., skin temperature, heart rate variability, inhale-exhale heart rate, respiration rate, oxygen saturation, pulse arrival time/pulse transit time, blood pressure, galvanic skin response, posture, movement, facial expression, voice tone and pitch, etc.,) and multiple psychological outputs (e.g., stress, appreciation, frustration, arousal, etc.,). The psychological outputs are aggregated to represent the subject's state of mind. Remedial action can then be taken in response to the subject's state of mind having been represented.

It should be understood that one of skilled in this art would readily understand various aspects of obtaining physiological signals from a subject using non-contact-based imaging method as disclosed in several of the incorporated references by Lalit K. Mestha, Edgar Bernal, Beilei Xu, and Survi Kyal. Such a person would also be readily familiar with obtaining physiological signals from a subject using contact-based methods. See: “Physiological Control Systems: Analysis, Simulation, and Estimation”, Wiley-IEEE Press (1999), ISBN-13: 978-0780334083, which is incorporated herein in its entirety by reference.

### Non-Limiting Definitions

A “subject” refers to a living being. Although the term “person” or “patient” may be used throughout this disclosure, it should be appreciated that the subject may be something other than a human such as, for example, a primate. Therefore, the use of such terms is not to be viewed as limiting the scope of the appended claims strictly to human beings with a respiratory function.

“Physiological features” also referred to as “physiological parameters” are obtained from the subject directly, are obtained from signals sensed from the subject, or are about a physiological condition of the subject. Physiological features can be any of: functional blood oxygen saturation, fractional blood oxygen saturation, flow-volume loops, expiratory reserve volume, inspiratory reserve volume, residual volume, vital capacity, inspiratory capacity, functional residual capacity, total lung capacity, tidal breathing, minute ventilation, respiration rate, and a breathing pattern of the subject, as are generally understood in the medical arts. Physiological features can further be any of: a Poincaré Plot of peak-to-peak pulse dynamics, a Pulse Harmonic Strength of at least a segment of the subject's cardiac signal, a frequency of the subject's normalized heartbeat, a peak-to-peak interval of at least a segment of the subject's cardiac signal, systolic and diastolic measurements, cardiac output, heart rate variability, blood pressure, blood vessel dilation over time, blood flow velocity, pulse rate, pulse amplitudes, temperature, as well as an electro-dermal response, a skin conductance response, a skin conductance level, a galvanic skin response, and skin resistance, as are also generally understood in the medical sensing arts. Physiological features can also be, for example, statistical features of physiological signals, signal amplitude, signal frequency, and one or more signal characteristics of the physiological signals, as are generally understood in the signal processing arts. In accordance with the teachings hereof, a vector containing multiple values of the physiological features is provided to a psychophysiological I/O model. It is to be noted that the physiological features provided to a psychophysiological I/O model could also be any one feature (i.e., a scalar) mentioned above.

“Physiological signals” are signals obtained by contact-based and non-contact-based sensing. Physiological signals can be any of: an electrocardiographic signal, a ballistocardiographic signal, an electroencephalographic (EEG) signal, an echocardiographic (ECG/EKG) signal, an electromyographic (EMG) signal, a phonocardiographic (PCG) signal, a videoplethysmographic (VPG) signal, a galvanic skin response (GSR) signal, a signal from a spot radiometer, and a signal from a thermometer, as such devices are generally understood. The physiological signals can also be eye movement, muscle twitch, voice tone, voice pitch, an audio signal of the subject's breathing pattern, and a signal representative of the subject's posture or facial expression. It should be appreciated that a physiological signal can be a reference signal such as, for instance, a reference cardiac signal or may be a user input.

“Contact-based sensing means” refers to systems and methods for obtaining physiological signals using one or more sensors which physically touch the subject or which are placed on a garment worn by the subject. Sensors are associated with any of a wide array of medical sensing devices which include, for example, an electrocardiographic device, a ballistocardiographic device, an electroencephalographic device, an echocardiographic device, an electromyographic device, a phonocardiographic device, and a galvanic skin response device, as such devices are generally understood in the arts.

“Non-contact-based sensing means” refers to obtaining physiological signals from a subject using systems and methods which do not require physical contact with the subject. Such methods include video-based sensing techniques as disclosed in several of the incorporated references. Video-based sensing techniques include any of: a monochrome video camera, a color video camera, a single-band infrared camera, a multi-band infrared camera in the thermal range, a multi-spectral camera, a hyperspectral camera, a hyperspectral infrared camera in the thermal range, and a hybrid video device comprising any combination hereof, as such video imaging devices are understood in the imaging arts.

A “psychophysiological model” is an input/output (I/O) model which receives, as input, a vector of physiological features and which generates, as output, psychological quantities. In one embodiment, the psychophysiological model comprises a plurality of fitting functions which are aggregated to obtain an aggregate output as follows:

$$y = {\sum }_{n = 1}^{m} {W}_{n} {f}_{n} ( \vec{u} ) , \qquad ( 1 )$$

where ƒₙ is the n^{th} fitting function (e.g., linear regression, least squares, splines, quadratic approximation, affine transform, feature vector, basis vector, etc.) each fitting at least one physiological feature to at least one psychological quantity, \vec{u}=(u₁, u₂, . . . , uⱼ) can be a vector comprising features obtained of the subject where j is the number of features, and Wₙ is a weight applied to the n^{th} function. For a survey of fitting functions, see: “Numerical Methods of Curve Fitting”, P. G. Guest (Author), Cambridge University Press, (2012), ISBN-13: 978-1107646957. See also, “Numerical Methods: Using MATLAB”, George Lindfield (Author), John Penny (Author), Academic Press, 3rd Ed. (2012), ISBN-13: 978-0123869425, both of which are incorporated herein in their entirety by reference.

In one embodiment, the psychophysiological model takes the following linear form which generates a vector of psychological quantities, as given by:

y=Aθ (2)

where A is a regression matrix A containing a form of a psychophysiological model, and θ is a matrix containing parameters of the psychophysiological model. In one embodiment, θ is given by:

$$θ = [ \begin{matrix}{M}_{10} & {M}_{20} & {M}_{30} \\ {M}_{11} & {M}_{21} & {M}_{31} \\ {M}_{12} & {M}_{22} & {M}_{32} \\ {M}_{13} & {M}_{23} & {M}_{33} \\ {M}_{14} & {M}_{24} & {M}_{34}\end{matrix} ] \qquad ( 3 )$$

where Mⱼₖ are different parameters.

Since matrix A may be a non-square matrix and thus not invertible, θ cannot be solved using matrix manipulation. Thus, we form a residue equation:

r=y−Aθ (4)

and then form the sum of the squares of the residues:

S=rr^{T}=(y−Aθ)(y−Aθ)^{T} (5)

Now minimize S by differentiating Eqn. (5) with respect to θ and equating the resulting equation to zero. This yields a standard least squares solution given by:

θ=(A^{T}A)⁻¹A^{T}y (6)

Eqn. (6) is used to obtain the parameter matrix representing the psychophysiology of the person (i.e., the state of mind estimation matrix).

“Pre-processing the physiological signals” means to perform any of: weighting, filtering, detrending, averaging, discarding, upsampling, down-sampling, smoothing, transforming, synchronizing, normalizing, and selecting batch of samples to represent the physiological signals. Pre-processing may be performed automatically and manually.

“Receiving a vector” is intended to be widely construed and includes: retrieving, capturing, acquiring, generating, or otherwise obtaining physiological features. Methods for generating a vector from a plurality of features are well established in the mathematical arts.

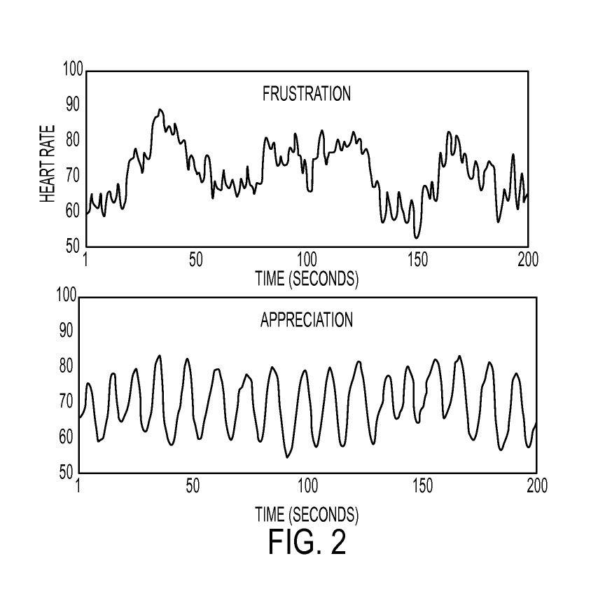

“Psychological quantities”, as used herein, refers to a state of the subject's mind. Example psychological quantities are fatigue, fear, stress, hunger, appreciation, alertness, frustration, anxiety, anger, happiness, arousal, drowsiness, to name a few. FIG. 2 shows two heart rate graphs for two different emotional states: frustration and appreciation. During frustration, heart rate variation is irregular and contain large fluctuations. Erratic and small heart beat fluctuations that occur with emotional dysregulation indicates that the sympathetic and the parasympathetic nervous systems are out of sync with each other. Generally during a typical rest period, heart rate during inhalation is high (e.g., 85 bpm) and exhalation is low (e.g., 75 bpm) with a difference of 10 bpm.

“Remedial action” is taken in response to the subject's state of mind having been represented using the teachings hereof to promote, avoid, or otherwise remedy the situation depending on the case. Such actions may take any of a variety of forms such as, for example, having the subject at rest or sending them home, assigning a different job function to that person, giving that person a break, rendering aid or assistance to that person, calling for professional help or assistance, providing medication to the person, providing feedback to the person, and the like. Remedial action may further be, for instance, automatically reducing vehicle speed while the person is driving or shutting down machinery being operated by the person. Remedial action may also comprise automatically changing lighting around the person as studies show that exposing a person to different lighting (e.g., blue) or to different colors (e.g., pink) can have a calming effect and potentially reduce stress.

“Communicating the psychological quantities” means to electronically transmit the output of the psychophysiological model to any of: a memory, a storage device, a smartwatch, a smartphone, a display, an iPad, a tablet-PC, a laptop, a workstation, and a remote device over a wired or wireless network. Such communication may take the form of signals.

It should be appreciated that the steps of “receiving”, “providing”, “communicating”, “aggregating”, “performing”, “pre-processing”, “weighting”, “filtering”, “detrending”, “averaging”, “discarding”, “upsampling”, “down-sampling”, “smoothing”, “transforming”, “synchronizing”, “normalizing”, “selecting”, and the like, as used herein, include the application of any of a variety of signal processing techniques as well as mathematical operations according to any specific context or for any specific purpose. It should be appreciated that such steps may be facilitated or otherwise effectuated by a microprocessor executing machine readable program instructions such that an intended functionality can be effectively performed.

### Example Flow Diagram

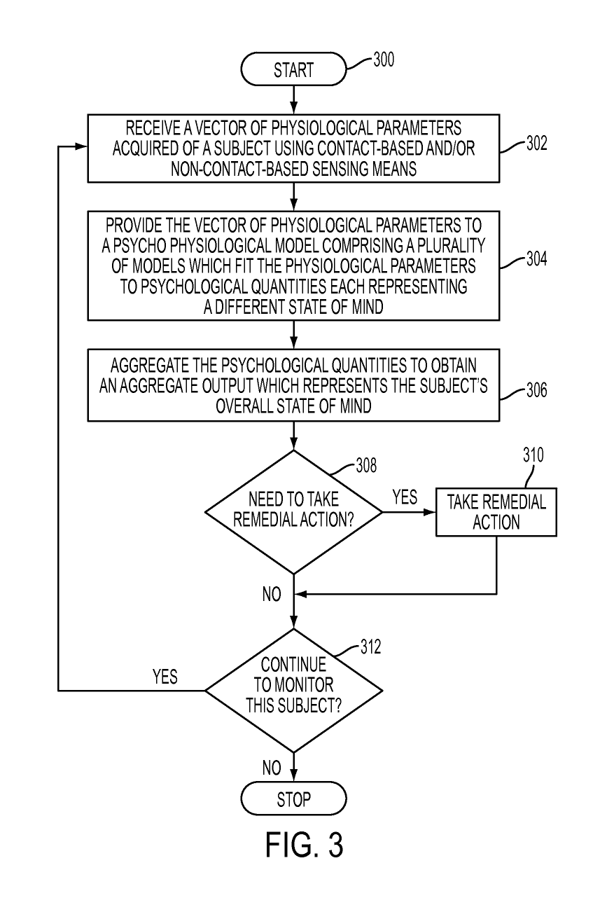

Reference is now being made to the flow diagram of FIG. 3 which illustrates one example embodiment of the present method for representing a person's state of mind from a plurality of physiological inputs. Flow processing begins at step 300 and immediately proceeds to step 302.

At step 302, receive a vector of physiological parameters of a subject using contact-based and/or non-contact-based sensing means.

At step 304, provide the vector of physiological parameters to a psychophysiological model comprising a plurality of models which fit the physiological parameters to psychological quantities each representing a different state of mind.

At step 306, aggregate the psychological quantities to obtain an aggregate output which represents of the subject's state of mind.

At step 308, a determination is made whether remedial action is to be taken. If so then, at step 310, take remedial action. As there are numerous scenarios for feedback/intervention, there is no “one size fits all” remedy. For example, in a call center environment where customer-agent interactions routinely occur, scripted dialogs can be more effectively managed by repeatedly assessing the subject's state of mind during working hours.

At step 312, a determination is made whether to continue to monitor this subject. If so then processing repeats with respect to step 302 wherein a next vector of physiological parameters for this subject is received for processing. Processing repeats in a similar manner. Otherwise, in this embodiment, further processing stops.

It should be appreciated that the flow diagrams depicted herein are illustrative. One or more of the operations in the flow diagrams may be performed in a differing order. Other operations may be added, modified, enhanced, or consolidated. Variations thereof are intended to fall within the scope of the appended claims.

### Block Diagram of Signal Processing System

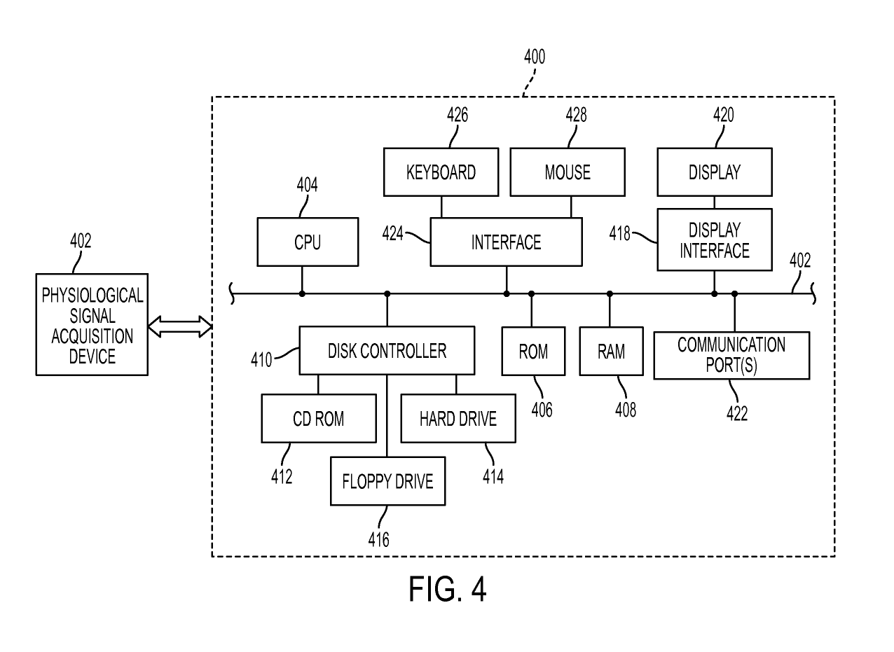

Reference is now being made to FIG. 4 which shows a functional block diagram of a special purpose for performing various aspects of the present method for representing a subject's state of mind as described with respect to the flow diagram of FIG. 3. Such a special purpose processor 400 is capable of executing machine readable program instructions for performing the methods disclosed herein.

In FIG. 4, communications bus 402 serves as an information highway interconnecting the other illustrated components of special purpose computer system 400. The special purpose computer incorporates a central processing unit (CPU) 404 capable of executing machine readable program instructions. The CPU is in communication with Read Only Memory (ROM) 406 and Random Access Memory (RAM) 408 which, collectively, constitute memory storage devices. Such memory may be used to store machine readable program instructions and other program data and results to sufficient to carry out any of the functionality described herein.

Disk controller 410 interfaces with one or more storage devices 414. Storage devices may comprise external memory, zip drives, flash memory, USB drives, memory sticks, or devices with removable media such as CD-ROM drive 412 and floppy drive 416. Such storage devices may be used to implement a database. Example computer readable media is, for example, a floppy disk, a hard-drive, memory, CD-ROM, DVD, tape, cassette, or other digital or analog media, or the like, which is capable of having embodied thereon a computer readable program, one or more logical instructions, or other machine executable codes or commands that implement and facilitate the function, capability, and methodologies described herein. Computer programs (also called computer control logic) may be stored in a main memory and/or a secondary memory. Computer programs may also be received via the communications interface. The computer readable medium is further capable of storing data, machine instructions, message packets, or other machine readable information, and may include non-volatile memory. Display interface 418 effectuates the display of information on display device 420 in various formats such as, for instance, audio, graphic, text, and the like. Interface 424 effectuates a user input via keyboard 426 and mouse 428. Such an interface is useful for a user to review and enter information about any of the displayed information in accordance with various embodiments hereof. Communication with external devices may occur using example communication port(s) 422. Such ports may be placed in communication with the Internet or an intranet, either by wired or wireless link. Example communication ports include modems, network cards such as an Ethernet card, routers, a PCMCIA slot and card, USB ports, and the like, capable of transferring data from one device to another. Also shown is Physiological Signal Acquisition Device 402 which may be any of the devices described herein capable of transferring acquired physiological signals or physiological parameters to the special purpose computer 400 via any of the communication ports. Such signals which may be digital, analog, electromagnetic, optical, infrared, or other signals capable of being transmitted and/or received by the communications interface. Such signals may be implemented using, for example, a wire, cable, fiber optic, phone line, cellular link, RF, or other signal transmission means presently known in the arts or which have been subsequently developed.

### VARIOUS EMBODIMENTS

One or more aspects of the methods described herein are intended to be incorporated in an article of manufacture, including one or more computer program products, having computer usable or machine readable media. The article of manufacture may be included on at least one storage device readable by machine architectures embodying executable program instructions capable of performing one or more aspects of the methods described herein. The article of manufacture may be shipped, sold, leased, or otherwise provided separately either alone or as part of an add-on, update, upgrade, or product suite.

It will be appreciated that the above-disclosed and other features and functions, or alternatives thereof, may be desirably combined into many other different systems or applications. Various presently unforeseen or unanticipated alternatives, modifications, variations, or improvements therein may become apparent and/or subsequently made by those skilled in the art which are also intended to be encompassed by the following claims. The teachings hereof may be partially or fully implemented in software using object or object-oriented software development environments that provide portable source code that can be used on a variety of computer, workstation, server, network, or other hardware platforms. One or more of the capabilities hereof can be emulated in a virtual environment utilizing specialized programs or may leverage off-the-shelf software. It will be appreciated that various of the above-disclosed and other features and functions, or alternatives thereof, may be combined into other systems or applications. Various presently unforeseen or unanticipated alternatives, modifications, variations, or improvements therein may become apparent and/or subsequently made by those skilled in the art which are also intended to be encompassed by the following claims.

The embodiments set forth are considered to be illustrative and not limiting. Changes to the above-described embodiments may be made without departing from the spirit and scope of the invention. The teachings hereof can be implemented in hardware or software using any known or later developed systems, structures, devices, and/or software by those skilled in the applicable art without undue experimentation from the functional description provided herein with a general knowledge of the relevant arts. The teachings of any printed publications including patents and patent applications, are each separately hereby incorporated by reference in their entirety.

What is claimed is:

1. A computer implemented method for representing a person's state of mind from a plurality of physiological parameters, comprising:
receiving a vector \vec{u}=(u₁, u₂, . . . , uⱼ) comprising features of at least one physiological signal obtained of a subject;
providing said feature vector to a psychophysiological model comprising a plurality of models which relate physiological features to psychological quantities, each representing a different state of mind; and
aggregating said psychological quantities to obtain an aggregate output representing said subject overall state of mind.

2. The method of claim 1, wherein said physiological signals are obtained by a wearable sensor associated with any of: an electrocardiographic device, a ballistocardiographic device, an electroencephalographic device, an echocardiographic device, an electromyographic device, a phonocardiographic device, and a galvanic skin response device.

3. The method of claim 1, wherein said physiological signals are obtained by non-contact-based sensing comprises any of: a monochrome video camera, a color video camera, a single-band infrared camera, a multi-band infrared camera in the thermal range, a multi-spectral camera, a hyperspectral camera, a hyperspectral infrared camera in the thermal range, and a hybrid video device comprising any combination hereof.

4. The method of claim 1, wherein said physiological signals comprise any of: an electrocardiographic signal, a ballistocardiographic signal, an electroencephalographic signal, an echocardiographic signal, an electromyographic signal, a phonocardiographic signal, a videoplethysmographic signal, an audio signal of said subject's breathing pattern, a signal of a galvanic response of said subject's skin, a signal from a spot radiometer, a signal from a thermometer, and a reference signal.

5. The method of claim 1, wherein said physiological features comprise any of: functional blood oxygen saturation, fractional blood oxygen saturation, flow-volume loops, expiratory reserve volume, inspiratory reserve volume, residual volume, vital capacity, inspiratory capacity, functional residual capacity, total lung capacity, tidal breathing, minute ventilation, respiration rate, and a breathing pattern of said subject.

6. The method of claim 1, wherein said physiological features comprise any of: a Poincaré Plot of peak-to-peak pulse dynamics, a pulse harmonic strength of at least a segment of said subject's cardiac signal, a frequency of said subject's normalized heartbeat, a peak-to-peak interval of at least a segment of said subject's cardiac signal, systolic and diastolic measurements, cardiac output, heart rate variability, blood pressure, blood vessel dilation over time, blood flow velocity, pulse rate, and pulse amplitudes.

7. The method of claim 1, wherein said physiological features comprise any of: an electro-dermal response, a skin conductance response, a skin conductance level, a galvanic skin response, skin resistance, and skin temperature.

8. The method of claim 1, wherein said physiological features comprise any of: eye movement, muscle twitch, voice tone, voice pitch, posture, facial expression, and a user input.

9. The method of claim 1, wherein said physiological features comprise any of: statistical features of a physiological signal, amplitude of a physiological signal, frequency of a physiological signal, and characteristics of a physiological signal.

10. The method of claim 1, wherein said psychological quantities comprises any of: fatigue, fear, stress, hunger, appreciation, alertness, frustration, anxiety, anger, happiness, arousal, and drowsiness.

11. The method of claim 1, further comprising pre-processing any of said physiological signals by any of:
weighting at least a segment of one of said physiological signals;
band pass filtering any of said signals to restrict frequencies of interest;
filtering any of said physiological signals to remove unwanted artifacts;
detrending said signals to remove low frequency and non-stationary components;
averaging any of said signals to obtain a composite physiological signal;
discarding at least a portion of any of said physiological signals;
upsampling any of said physiological signals to a standard sampling frequency;
down-sampling any of said signals to a standard sampling frequency;
smoothing at least a segment of any of said physiological signals;
transforming any of said physiological signals into an alternate domain;
synchronizing any of said physiological signals with respect to time;
normalizing any of said physiological signals to unit variance, and
selecting batch of samples to represent said physiological signals.

12. The method of claim 1, further comprising communicating said subject's overall state of mind to any of: a memory, a storage device, a smartwatch, a smartphone, a display, an iPad, a tablet-PC, a laptop, a workstation, and a remote device over a network.

13. The method of claim 1, wherein said physiological signals are streaming signals and said subject's state of mind is represented in real-time.

14. The method of claim 1, further comprising taking remedial action with respect to said subject in response to said subject's state of mind having been represented.

15. The method of claim 14, wherein remedial action comprises any of: having said subject rest, sending said subject home, assigning a different job function to said subject, giving said subject a break, rendering assistance to said subject, calling for medical help for said subject, providing medication to said subject, reducing vehicle speed while driving, changing lighting, and initiating an alert.

16. A system for representing a person's state of mind from a plurality of physiological parameters, the system comprising:
a storage device; and
a processor in communication with said storage device, said processor executing machine readable instructions for:
receiving a vector \vec{u}=(u₁, u₂, . . . , uⱼ) comprising features of at least one physiological signal obtained of a subject;
providing said feature vector to a psychophysiological model comprising a plurality of models which relate physiological features to psychological quantities, each representing a different state of mind; and
aggregating said psychological quantities to obtain an aggregate output representing said subject overall state of mind.

17. The system of claim 16, wherein said physiological signals are obtained by a wearable sensor associated with any of: an electrocardiographic device, a ballistocardiographic device, an electroencephalographic device, an echocardiographic device, an electromyographic device, a phonocardiographic device, and a galvanic skin response device.

18. The system of claim 16, wherein said physiological signals are obtained by non-contact-based sensing comprises any of: a monochrome video camera, a color video camera, a single-band infrared camera, a multi-band infrared camera in the thermal range, a multi-spectral camera, a hyperspectral camera, a hyperspectral infrared camera in the thermal range, and a hybrid video device comprising any combination hereof.

19. The system of claim 16, wherein said physiological signals comprise any of: an electrocardiographic signal, a ballistocardiographic signal, an electroencephalographic signal, an echocardiographic signal, an electromyographic signal, a phonocardiographic signal, a videoplethysmographic signal, an audio signal of said subject's breathing pattern, a signal of a galvanic response of said subject's skin, a signal from a spot radiometer, a signal from a thermometer, and a reference signal.

20. The system of claim 16, wherein said physiological features comprise any of: functional blood oxygen saturation, fractional blood oxygen saturation, flow-volume loops, expiratory reserve volume, inspiratory reserve volume, residual volume, vital capacity, inspiratory capacity, functional residual capacity, total lung capacity, tidal breathing, minute ventilation, respiration rate, and a breathing pattern of said subject.

21. The system of claim 16, wherein said physiological features comprise any of: a Poincaré Plot of peak-to-peak pulse dynamics, a pulse harmonic strength of at least a segment of said subject's cardiac signal, a frequency of said subject's normalized heartbeat, a peak-to-peak interval of at least a segment of said subject's cardiac signal, systolic and diastolic measurements, cardiac output, heart rate variability, blood pressure, blood vessel dilation over time, blood flow velocity, pulse rate, and pulse amplitudes.

22. The system of claim 16, wherein said physiological features comprise any of: an electro-dermal response, a skin conductance response, a skin conductance level, a galvanic skin response, skin resistance, and skin temperature.

23. The system of claim 16, wherein said physiological features comprise any of: eye movement, muscle twitch, voice tone, voice pitch, posture, facial expression, and a user input.

24. The system of claim 16, wherein said physiological features comprise any of: statistical features of a physiological signal, amplitude of a physiological signal, frequency of a physiological signal, and characteristics of a physiological signal.

25. The system of claim 16, wherein said psychological quantities comprises any of: fatigue, fear, stress, hunger, appreciation, alertness, frustration, anxiety, anger, happiness, arousal, and drowsiness.

26. The system of claim 16, further comprising pre-processing any of said physiological signals by any of:
weighting at least a segment of one of said physiological signals;
band pass filtering any of said signals to restrict frequencies of interest;
filtering any of said physiological signals to remove unwanted artifacts;
detrending said signals to remove low frequency and non-stationary components;
averaging any of said signals to obtain a composite physiological signal;
discarding at least a portion of any of said physiological signals;
upsampling any of said physiological signals to a standard sampling frequency;
down-sampling any of said signals to a standard sampling frequency;
smoothing at least a segment of any of said physiological signals;
transforming any of said physiological signals into an alternate domain;
synchronizing any of said physiological signals with respect to time;
normalizing any of said physiological signals to unit variance, and
selecting batch of samples to represent said physiological signals.

27. The system of claim 16, further comprising communicating said subject's overall state of mind to any of: a memory, a storage device, a smartwatch, a smartphone, a display, an iPad, a tablet-PC, a laptop, a workstation, and a remote device over a network.

28. The system of claim 16, wherein said physiological signals are streaming signals and said subject's state of mind is represented in real-time.

29. The system of claim 16, further comprising taking remedial action with respect to said subject in response to said subject's state of mind having been represented.

30. The system of claim 29, wherein remedial action comprises any of: having said subject rest, sending said subject home, assigning a different job function to said subject, giving said subject a break, rendering assistance to said subject, calling for medical help for said subject, providing medication to said subject, reducing vehicle speed while driving, changing lighting, and initiating an alert.
